(19) United States
(12) Patent Application Publication
(10) Pub. No.: US 2021/0268190 A1
(43) Pub. Date: Sep. 2, 2021

(54) MEDICAMENT DELIVERY DEVICE

(71) Applicant: SHL MEDICAL AG, ZUG (CH)

(72) Inventor: Erika André, Saltsjö-Boo (SE)

(21) Appl. No.: 16/972,508

(22) PCT Filed: Jun. 25, 2019
(86) PCT No.: PCT/EP2019/066729

§ 371 Date: Dec. 4, 2020

(30) Foreign Application Priority Data
Jul. 20, 2018 (EP) 18184837.5

(51) Int. Cl. A61M 5/20; A61M 5/315

(52) U.S. Cl. CPC A61M 5/2033; A61M 2005/2073; A61M 5/31585

(57) ABSTRACT

A medicament delivery device is presented having a housing that is arranged to accommodate a medicament container, a drive unit operable of exerting a force on the medicament container for expelling a dose of medicament, wherein the drive unit may comprise variable numbers of drive springs depending on force requirements for expelling the dose of medicament.

### CROSS REFERENCE TO RELATED APPLICATIONS

The present application is a U.S. National Phase application pursuant to 35 U.S.C. § 371 of International Application No. PCT/EP2019/066729 filed Jun. 25, 2019, which claims priority to European Patent Application No. 18184837.5 filed Jul. 20, 2018. The entire disclosure contents of these applications are herewith incorporated by reference into the present application.

### TECHNICAL AREA

The present application relates to a medicament delivery device capable of handling different force requirements regarding dose delivery.

### BACKGROUND

In the field of medicament delivery devices in particular for self-administration there are frequent proposals by the pharma industry to have medicament delivery devices that are capable of handling not only one specific medicament container but several different medicament containers regarding both volume and the viscosity of the medicament. These differences lead to different force requirements that could vary quite substantially. This in turn would mean that a drive spring in the medicament delivery device has to be dimensioned for the largest force requirements which then would mean unnecessarily high forces when the requirements are much lower, which in turn would risk breakage of the medicament containers.

Some earlier medicament delivery devices have elaborated with more than one spring to handle force requirements. For instance document WO 9421316 discloses a medicament delivery device provided with two coil springs that work in conjunction with each other. This solution is a substitute for a large spring when large forces are required, so that the device does not become unnecessarily large. The document does however not discuss varying force requirements due to different medicament container volumes or different viscosities. There is thus a need for solutions that can handle a range of force requirements in the same medicament delivery device.

Another aspect regarding drive springs is that some devices utilize clock springs when large forces are required. One such device is disclosed in WO 2012/173553 in which a clock spring is used for driving a plunger rod so that a dose of medicament is expelled through an injection needle. In order to perform the penetration of the injection a container driver with a separate spring is used to drive a medicament container holder. Thus an automatic penetration is performed. The device works well for its intended function but because of the automatic penetration, the number of components increase and the complexity of the device increases.

### SUMMARY

In the present disclosure, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site.

The aim of the present application is to remedy the drawbacks of the state of the art device. According to the present application, it is solved by a medicament delivery device according to the features of the independent patent claims. Preferable embodiments of the present application form the subject of the dependent patent claims.

According to a main aspect, a medicament delivery device is arranged comprising a housing, wherein the housing is arranged to accommodate a medicament container. The housing further comprises a drive unit operable of exerting a force on the medicament container for expelling a dose of medicament. According to one important aspect, the drive unit may comprise any of one to several drive springs depending on force requirements for expelling the dose of medicament. With this solution, the drive unit comprises variable numbers of drive springs, which numbers of drive springs is depending on the force requirement for expelling the dose of medicament regarding to different volume or viscosity of different medicament, it is possible to “customize” the force from the drive unit depending on the requirements for expelling a dose from a specific medicament container. Thus a wide range of forces can be handled with the same device with different number of drive springs.

According to one aspect, the drive spring or drive springs may comprise clock springs. An advantage with that is that clock springs do not add so much to the overall length of the medicament delivery device as conventional coiled compression springs do since they are positioned with their windings transversal to a longitudinal axis of the medicament delivery device and thus it is the width of the clock spring that affects the length of the medicament delivery device. Since the width of clock springs are smaller than the length of a compression spring for a comparable force output, a number of clock springs do not affect the overall length of the medicament delivery device in any substantial way.

The drive unit may comprise a rotatable driver that the drive spring or drive springs are operably connected to. In this case, one end of drive spring(s) is(are) attached to the driver. Further the drive unit may comprise a plunger rod operably connected to the driver.

Further a threaded drive nut may be provided, cooperating with threads on the plunger rod for driving the plunger rod to expel a dose of medicament when the driver and the plunger rod are rotated.

The medicament delivery device may also comprise a locking mechanism for releasibly locking the driver, and an actuator may be operably connected to the locking mechanism for releasing the driver upon activation of the actuator. More precisely the locking mechanism may comprise a mechanical stop on the driver and a corresponding mechanical stop on the actuator wherein the actuator is movable from a first position where the mechanical stops engage to a second position where the mechanical stops are out of engagement, releasing the driver.

According to a further aspect, the medicament delivery device may comprise an activator operably connected to the actuator for manually operating the actuator and according one favourable aspect, the activator may comprise a movable medicament delivery member guard arranged at a proximal area of the medicament delivery device.

The medicament delivery device may further comprise a rotator comprising a locking mechanism operably connected to the medicament delivery member guard such that movement of the medicament delivery member guard in a distal direction will rotate the rotator such that subsequent movement of the medicament delivery member guard in the proximal direction during removal of the medicament delivery device after dose delivery will cause the medicament delivery member guard to interact with the locking mechanism whereby the medicament delivery member guard is locked in an extended position.

The medicament delivery device may further comprise a holding element for the medicament container that is fixed inside the medicament delivery device. With this solution there is no automatic penetration but a manual penetration since the medicament container with its medicament delivery member is fixed in the medicament delivery device. In this regard, the holding element may form an integral part of the housing. As an alternative the holding element may comprise a medicament container holder fixedly attached inside the housing.

According to another aspect, the medicament delivery device may further comprise a holding element for the medicament container that is arranged inside the medicament delivery device; and is axially movable in relation to the medicament delivery device. With this solution, an automatic penetration function is performed. In this regard, the holding element may comprise a penetration stop and the housing may comprise a counter stop, so that the distance of the automatic penetration can be determined.

These and other aspects of, and advantages with, the present disclosure will become apparent from the following detailed description of the present disclosure and from the accompanying drawings.

### BRIEF DESCRIPTION OF DRAWINGS

In the following detailed description of the present disclosure, reference will be made to the accompanying drawings, of which

FIG. 1 is an exploded view of a medicament delivery device according to the present application,

FIG. 2 is a perspective view of the medicament delivery device according to FIG. 1,

FIG. 3 is a cross-sectional view of the medicament delivery device according to FIG. 1 taken along a longitudinal axis,

FIG. 4 is a detailed view of components and functions of the medicament delivery device of FIG. 1,

FIG. 5 is a detailed view of components and functions of the medicament delivery device of FIG. 1,

FIG. 6 is a detailed view of components and functions of the medicament delivery device of FIG. 1,

FIG. 7 is a detailed view of components and functions of the medicament delivery device of FIG. 1,

FIG. 8 is a detailed view of components and functions of the medicament delivery device of FIG. 1,

FIG. 9 is a detailed view of components and functions of the medicament delivery device of FIG. 1,

FIG. 10 is a detailed view of components and functions of the medicament delivery device of FIG. 1,

FIG. 11 is a detailed view of components and functions of the medicament delivery device of FIG. 1,

FIG. 12 is a detailed view of components and functions of the medicament delivery device of FIG. 1,

FIG. 13 is a detailed view of components and functions of the medicament delivery device of FIG. 1, and

FIG. 14 is a detailed view of components and functions of the medicament delivery device of FIG. 1.

### DETAILED DESCRIPTION

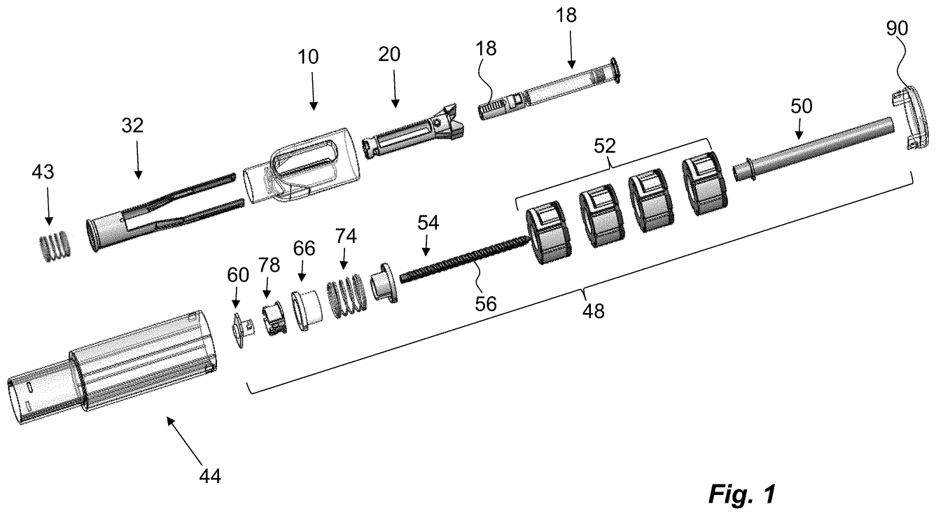

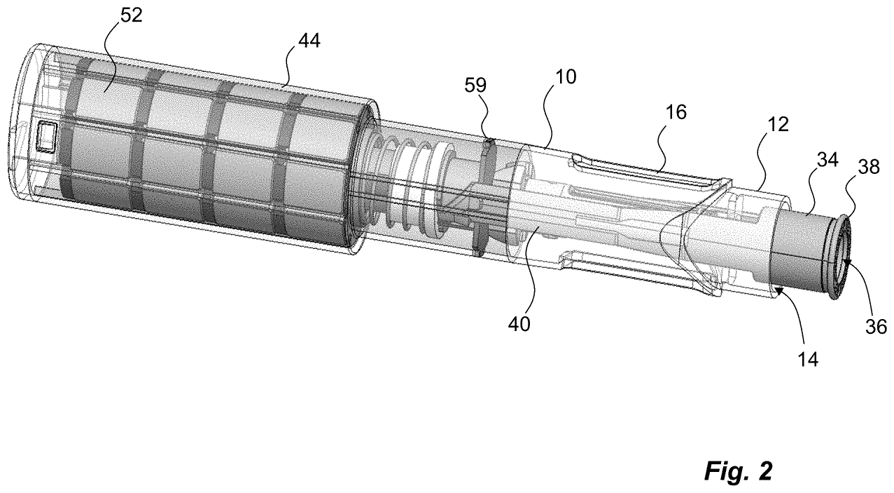

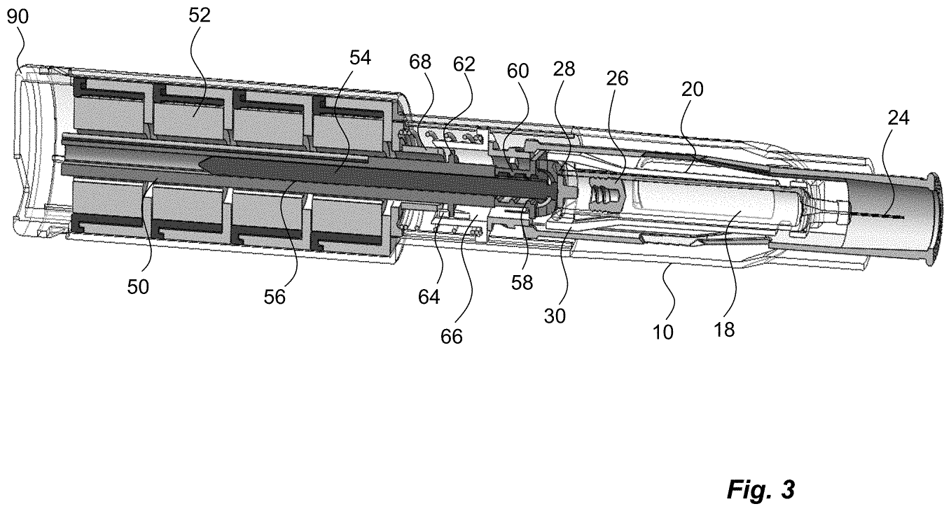

The medicament delivery device shown in the drawings comprises a generally tubular proximal housing part 10. The proximal housing part 10 is arranged with a proximal area 12 that may have a somewhat smaller diameter, where a safety cap (not shown) may be connected to, FIGS. 1 and 2. The proximal end of the proximal housing part 10 may be arranged with a central passage 14. The side surfaces of the proximal housing part 10 may be arranged with openings or windows 16, through which a medicament container 18 may be viewed, FIGS. 1 and 3. The medicament container 18 may be placed in a suitable holding element such as a medicament container holder 20, which container holder is placed and is fixedly supported inside the proximal housing part 10, FIG. 3. It is however to be understood that the holding element may form an integral part of the housing or any other component fixed inside the medicament delivery device. The medicament container 18 is in the shown embodiment a syringe having a rigid needle shield or RNS 22 covering a medicament delivery member in the form of an injection needle 24, FIG. 1. The tubular body of the medicament container 18 is arranged with a movable stopper 26. The distal end of the medicament container 18 is arranged with a radially extending flange 28 that is seated in a distal seat 30 of the medicament container holder 20.

In the central passage 14 of the proximal housing part 10, a medicament delivery member guard 32 may be movably or slidably arranged and protruding out of the proximal end of the proximal housing part 10, FIG. 2. The medicament delivery member guard 32 may further comprise a tubular body 34 having a central passage 36. The central passage 36 may be surrounded by a radially outwardly extending flange 38 or ledge, providing a proximally directed contact surface for the medicament delivery member guard 32. Two distally directed arms 40 are further attached to a distal end surface of the body 34. The arms 40 are arranged with inwardly directed protrusions 42, the function of which will be described below. The medicament delivery member guard 32 is urged in the proximal direction by a medicament delivery member guard spring 43, FIG. 1, acting between a distal surface of the medicament delivery member guard 32 and a proximal surface of the proximal housing part 10.

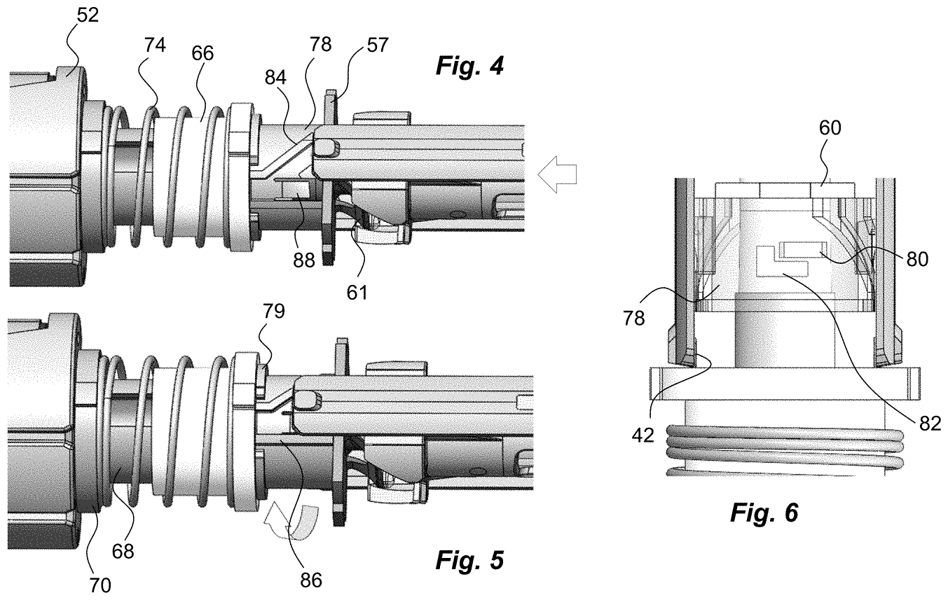

To the proximal housing part 10 a distal housing part 44 is attached, FIG. 2. In this regard, the proximal end of the distal housing part 44 may be arranged with a section 46 with a diameter that fits into the distal area of the proximal housing part 10. The distal housing part 44 is further arranged with a drive unit or power pack 48, FIG. 1. The drive unit 48 comprises an elongated tubular driver 50. The outer surface of the driver 50 is arranged with attachment elements that are capable of holding inner ends of a number of clock springs 52, FIGS. 1 and 3. The outer ends of the clock springs 52 are attached to fixed points of the medicament delivery device such as attachment elements on the inner surface of the distal housing part 44. Inside the driver 50 a plunger rod 54 is arranged. The inner surface of the driver 50 and the outer surface of the plunger rod 54 are arranged with guide elements such that the plunger rod 54 is slidable but rotationally locked in relation to the driver 50. The outer surface of the plunger rod 54 is further arranged with threads 56, FIG. 3, which threads 56 are to cooperate with threads 58 of a drive nut 60 that is fixedly attached to an element or a part of the stationary housing. In the embodiment shown the drive nut 60 is provided with radially extending protrusions 57, FIG. 4, that are to engage with recesses or cut-outs 59, FIG. 1, in the proximal housing part 10. The drive nut 60 is further arranged with proximally directed resilient support elements 61, FIG. 4, that are resiliently acting on the distal end of the medicament container 18 for reducing or minimizing movement of the medicament container 18 in the longitudinal direction; also, the resilient support elements 61 is able to compensate different length of different medicament containers, so that variable types of the medicament containers are compatible.

The driver 50 is further arranged with outwardly directed protrusions or ledges 62 at a proximal area thereof. The protrusions are to interact with longitudinally extending ledges 64 on an inner surface of a generally tubular actuator 66, FIG. 3. The actuator 66 is in turn guided in the longitudinal direction by a generally tubular seat 68, FIGS. 3 and 4. The seat 68 is arranged with an outwardly directed flange 70 that is abutting the most proximal of the springs of the power pack. The flange 70 is further arranged with circumferentially extending grooves 72, FIG. 8, on the proximal surface thereof. An actuator spring 74 is then provided between the grooves 72 of the flange 70 of the seat 68 and distally directed surface 76 of the actuator 66 so as to urge the actuator 66 in the proximal direction.

Proximal of the actuator 66 is a generally tubular rotator 78, FIGS. 4 and 5, having an outwardly directed flange 79 that is in contact with a proximal end surface of the actuator 66. The rotator 78 should be arranged rotatable but axially locked in relation to the device. The rotator 78 could be axially locked by a transversal ledge 80 on an inner surface of the rotator cooperating with a transversal or even L-shaped ledge 82 on an outer surface of the drive nut 60 as seen in FIG. 6. The rotator 78 is provided with inclined ledges 84 on its outer surface, FIG. 4, which inclined ledges 84 are to interact with the inwardly directed protrusions 42 of the medicament delivery member guard 32. The rotator 78 is further arranged with longitudinally extending ledges 86. A generally wedge-shaped tongue 88 that is flexible in the generally radial direction is arranged adjacent the longitudinal ledge 86, FIGS. 5 and 6.

Finally the distal end of the distal housing part may be arranged with an end lid 90 or the like, FIGS. 1 and 3. The end lid may be of a transparent material wherein the distal end of the driver may be arranged with a visual indicator that can indicate to a user when the device is in operation.

The device is intended to function as follows. Depending on the requirements of the medicament inside the medicament container and on the volume of the medicament container, different force requirements are put on the medicament delivery device. The medicament delivery device shown is thus arranged with a kind of modular force system. As seen in FIG. 3, the number of springs 52 used could be varied from 1 to 4 within the same device. Thus the device can handle a wide range of force requirements by varying the number of springs 52. If fewer than 4 springs are used, the unused space may then be occupied by adapters or dummies ensuring that the springs used cannot move or rattle inside the housing.

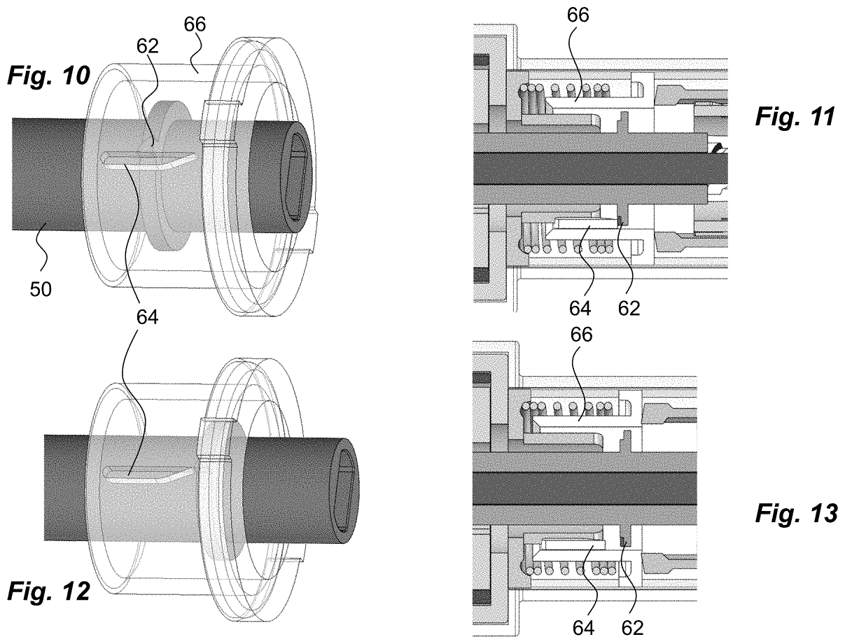

The device is most likely delivered to a user with a preselected number of springs 52 and preselected medicament container 18. The springs 52 are also preferably pre-tensioned whereby the medicament delivery device is ready for use. The springs 52 are then held pre-tensioned in that the protrusions 62 of the driver 50 are in contact with side surfaces of the longitudinal ledges 64 of the actuator 66 as seen in FIGS. 10 and 11, preventing rotation of the driver 50 and thus the plunger rod 54.

The user may now press the proximal end of the medicament delivery device against a dose delivery site. This will cause the medicament delivery member guard 32 that is protruding from the proximal end of the housing to be moved into the proximal housing part 10. The distal arms 40 of the medicament delivery member guard 32 will then move distally in relation to the rest of the medicament delivery device. This in turn will cause the inwardly directed protrusions 42 of the arms 40 to come in contact with the inclined ledges 84 of the rotator 78, FIG. 5, whereby the rotator 78 will rotate a certain angle so that the protrusions 42 are aligned with the wedge-shaped tongues 88 of the rotator 78 as seen in a longitudinal direction. Since the medicament container holder with the medicament container is fixed and non-movable inside the housing, a manual penetration or insertion of the medicament delivery member in the dose delivery site is performed.

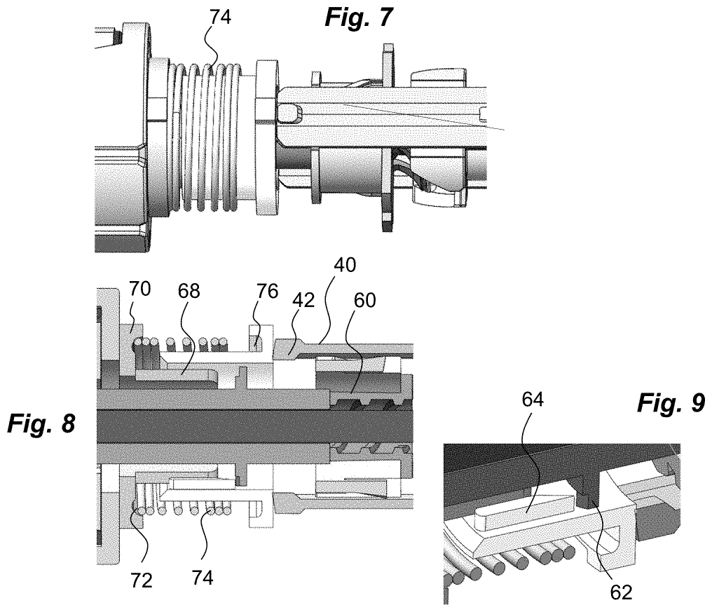

The distal ends of the arms 40 of the medicament delivery member guard 32 are now coming in contact with a proximally directed surface of the actuator 66, FIGS. 7 and 8, so that the actuator 66 will be moved in the distal direction against the force of the actuator spring 74. The movement of the actuator 66 will now cause the longitudinal ledges 64 to be moved out of engagement with the protrusions 62 of the driver 50, FIGS. 9, 12, 13, whereby it is released. The force from the spring or springs 52 can now rotate the driver 50 and thus the plunger rod 54 due to the rotational lock between the two elements. The rotation of the plunger rod 54 will then cause it to move in the proximal direction due to the threads 56 of the plunger rod 54 interacting with the threads 58 of the rotationally fixed drive nut 60. The movement in the proximal direction of the plunger rod 54 will cause it to act on the stopper 26 of the medicament container 18 to move it in the proximal direction and to expel a dose of medicament through the medicament delivery member 24 at the dose delivery site.

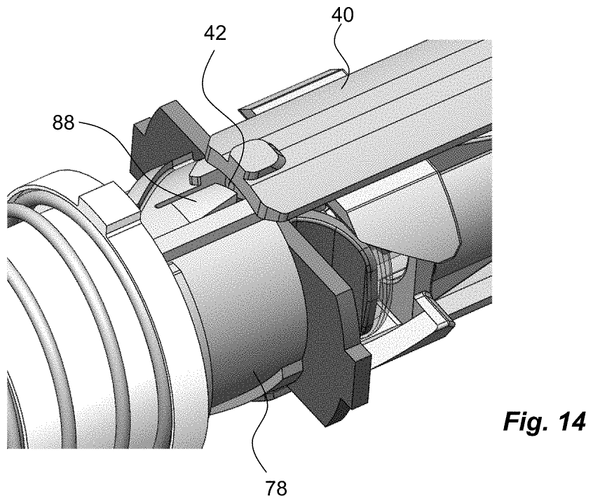

When the dose delivery sequence has ended, the medicament delivery device can be removed from the dose delivery site. The medicament delivery member guard 32 is now urged in the proximal direction to its initial position by the medicament delivery member guard spring 43. The movement of the medicament delivery member guard 32 will cause its protrusions 42 to move in the proximal direction so that they pass over the wedge-shaped tongues 88 of the rotator, FIG. 14, which will then cause the medicament delivery member guard 32 to be locked in the extended position, covering the medicament delivery member 24, and in turn preventing accidental injuries on the used medicament delivery member 24. The medicament delivery device may now be safely discarded.

1-14. (canceled)

15. A medicament delivery device comprising:
a housing that is arranged to accommodate a medicament container; and
a drive unit operable of exerting a force on the medicament container for expelling a dose of medicament,
wherein the drive unit comprises variable numbers of drive springs depending on force requirements for expelling the dose of medicament.

16. The medicament delivery device according to claim 15, wherein the drive spring or drive springs comprise clock springs.

17. The medicament delivery device according to claim 15, wherein the drive unit comprises a rotatable driver that the variable numbers of drive springs are operably connected to.

18. The medicament delivery device according to claim 17 wherein the drive springs comprise clock springs, where one end of any of one to several of the clock springs is attached to the driver.

19. The medicament delivery device according to claim 15, wherein the drive unit comprises a plunger rod operably connected to the driver.

20. The medicament delivery device according to claim 19, comprising a threaded drive nut cooperating with threads on the plunger rod for driving the plunger rod to expel a dose of medicament when the driver and the plunger rod are rotated.

21. The medicament delivery device according to claim 15, comprising a locking mechanism for releasably locking the driver, and an actuator operably connected to the locking mechanism for releasing the driver upon activation of the actuator.

22. The medicament delivery device according to claim 21, wherein the locking mechanism comprises a mechanical stop on the driver and a corresponding mechanical stop on the actuator wherein the actuator is movable from a first position where the mechanical stops engage to a second position where the mechanical stops are out of engagement, releasing the driver.

23. The medicament delivery device according to claim 21, comprising an activator operably connected to the actuator for manually operating the actuator.

24. The medicament delivery device according to claim 23, wherein the activator comprises a movable medicament delivery member guard arranged at a proximal area of the medicament delivery device.

25. The medicament delivery device according to claim 24, comprising a rotator comprising a locking mechanism operably connected to the medicament delivery member guard such that movement of the medicament delivery member guard in a distal direction will rotate the rotator such that subsequent movement of the medicament delivery member guard in the proximal direction during removal of the medicament delivery device after dose delivery will cause the medicament delivery member guard to interact with the locking mechanism whereby the medicament delivery member guard is locked in an extended position.

26. The medicament delivery device according to claim 15, further comprising a holding element for the medicament container that is fixed inside the medicament delivery device.

27. The medicament delivery device according to claim 26, wherein the holding element forms an integral part of the housing.

28. The medicament delivery device according to claim 27, wherein the holding element comprises a medicament container holder fixedly attached inside the housing.

29. A medicament delivery device comprising:
a housing that is arranged to accommodate a medicament container; and
a drive unit operable of exerting a force on the medicament container for expelling a dose of medicament,
wherein the drive unit comprises a driver and a plurality of clock springs, where a first end of each clock spring is fixed relative to the housing and a second end of each clock spring is operatively connected to the driver such that movement of the second ends causes the driver to rotate and expel the dose of medicament.

30. The medicament delivery device of claim 29 wherein the clock springs are pre-tensioned.

31. The medicament delivery device according to claim 29 further comprising: a locking mechanism that releasably locks the driver;
an actuator operably connected to the locking mechanism that releases the driver upon activation of the actuator; and
an activator operably connected to the actuator for manually operating the actuator.

32. The medicament delivery device according to claim 30, wherein the drive unit further comprises a plunger rod operably connected to the driver.

33. The medicament delivery device according to claim 32, comprising a threaded drive nut cooperating with threads on the plunger rod for driving the plunger rod to expel a dose of medicament when the driver and the plunger rod are rotated.

34. The medicament delivery device according to claim 31, wherein the activator comprises a movable medicament delivery member guard arranged at a proximal area of the medicament delivery device.
